(19) United States
(12) Patent Application Publication
(10) Pub. No.: US 2002/0143397 A1
(43) Pub. Date: Oct. 3, 2002

(54) Compliant artificial lung for extrapulmonary gas transfer

(76) Inventor: Ludwig K. von Segesser, Lausanne (CH)

Correspondence Address: MINTZ LEVIN COHN FERRIS, GLOVSKY AND POPEO PC, Suite 400, 11911 Freedom Drive, Reston, VA, 20190, US

(21) Appl. No.: 10/109,682

(22) Filed: Apr. 1, 2002

Related U.S. Application Data
(60) Provisional application No. 60/280,868, filed on Apr. 2, 2001.

(51) Int. Cl. A61F 2/20; A61F 2/54; A62B 7/10; A62B 23/02

(52) U.S. Cl. 623/9; 128/205.28; 623/66.1

(57) ABSTRACT

A compliant artificial lung for extrapulmonary gas transfer comprises a membrane oxygenator adapted to be disposed within a flexible housing or bladder. The bladder defines a hollow chamber for receiving blood introduced via a blood inlet nozzle. The membrane oxygenator comprises a bundle of microporous or non-porous, hollow fibers adapted to transport an oxygen-rich gas. Venous (unoxygenated) blood is introduced into the bladder through the blood inlet nozzle and flows through spaces between (and across the surfaces of) the various fibers comprising the fiber bundle. The surface contact between the venous blood and the microporous fibers of the bundle facilitates the gas-exchanging function of transporting oxygen to the blood while removing carbon dioxide. This aids in transforming venous blood into arterial blood. The compliant artificial lung is sufficiently flexible to withstand the volume, pressure, and flow-rate characteristics of blood pumped within a natural range of the stroke volume of the heart, such that it may simulate the function of an anatomical lung by accepting pulsatile blood flow from the heart, while providing a continuous flow of oxygenated blood as output.

### CROSS-REFERENCE TO RELATED APPLICATIONS

This Application claims priority from U.S. Provisional Patent Application Serial No. 60/280,868 filed Apr. 2, 2001, which is incorporated herein by reference.

### FIELD OF THE INVENTION

This invention relates generally to methods and apparatus for blood oygenation. This invention relates more particularly to an artificial lung that is sufficiently compliant to withstand the volume, pressure, and flow-rate characteristics of blood pumped within a natural range of the stroke volume of the heart, such that it may simulate the function of an anatomical lung by accepting pulsatile blood flow from the heart, while providing a continuous flow of oxygenated blood as output.

### BACKGROUND OF THE INVENTION

Respiratory failure may generally be defined as the inability of the lungs to adequately perform the gas-exchanging function of adding oxygen to the blood, while removing carbon dioxide from the blood.

Various methods and apparatus have been developed to assist the lungs during respiratory failure. In a cardiopulmonary bypass (CPB) procedure, for example, a heart/lung machine (i.e., a combination blood pump and blood oxygenator) may be used in an operating room to provide total support of heart and lung function during cardiac operations. A technique known as extracorporeal membrane oxygenation (ECMO) may also be used, which often entails the use of a pump, membrane oxygenator, and heat exchanger. Additional techniques, such as extracorporeal lung assist (ECLA) and extracorporeal carbon dioxide (CO₂) removal (ECCOR), are also known. Each of the aforementioned techniques typically requires an extracorporeal blood path or, in other words, a blood path situated or occurring outside of the body. In addition, many of these techniques also require a pump to maintain the flow of blood through an oxygenator, or other suitable, artificial gas-exchange device.

Alternative approaches for assisting the lungs during respiratory failure have focused on methods and apparatus for achieving intracorporeal, extrapulmonary gas transfer. One approach, for example, involves the implantation of an intravascular gas exchanger into the vasculature of the body. Although significant gas transfer has been demonstrated with such devices, their overall performance is often limited by the total membrane surface area (for gas exchange) which can be implanted into the vasculature of the body. Additionally, the pressure gradient that may be generated by intravascular gas exchangers may act to limit the passive return of the blood to the heart.

These and other drawbacks exist.

### SUMMARY OF THE INVENTION

The invention solving these and other problems relates to a compliant artificial lung designed to simulate the function of an anatomical lung by accepting pulsatile blood flow from the heart while providing a continuous flow of oxygenated blood as output.

According to an embodiment of the invention, the compliant artificial lung may comprise a membrane oxygenator adapted to be disposed within a flexible housing or bladder. The bladder may define a hollow chamber for receiving blood from the heart that is introduced via a blood inlet nozzle. The bladder may further be fabricated from any suitable elastic material (e.g., silicone, polyurethane, medical grade rubber, latex, or others) having sufficient flexibility and elasticity to withstand the volume, pressure, and flow-rate characteristics of blood pumped within a natural range of the stroke volume of the heart. The range of cardiac output for a human heart (including both adults and children) may average roughly between one-hundred milliliters of blood per minute and twelve liters of blood per minute. As such, the bladder may be designed to absorb or withstand, as an average, a flow of approximately one-hundred milliliters of blood to twelve liters of blood per minute without providing any appreciable resistance for the heart. It should be recognized that these values are provided as a guideline when designing the durability of the bladder, and should not be viewed as limiting.

According to an embodiment of the invention, the membrane oxygenator adapted to be disposed within the bladder may comprise a bundle of microporous or non-porous, hollow fibers for transporting an oxygen-rich gas. The fibers, which may be comprised of any gas-permeable, biocompatible material, may be wound about a hollow core in a spiral fashion so as to form a plurality of layers. In particular, the direction of the spiral winding of the fibers may change, layer by layer, to create a mesh of fibers arranged at oblique angles. Winding the fibers in such a manner results in the creation of vacant spaces between the fibers that may permit the flow of blood therethrough. In addition, the mesh of fibers resulting from the spiral winding may also act to resist the inward pressure that a flow of blood may exert on the fibers.

According to various embodiments of the invention, additional arrangements, geometries, or configurations of hollow fibers may also be used. For example, rather than winding hollow fibers about a hollow core in a spiral fashion, square or triangular sheets of fibers, or other shaped sheets of fibers, may be used. Other configurations may exist.

According to an embodiment of the invention, an oxygen-rich gas may be introduced into the fiber bundle by an external gas blender or other source. The gas may then travel through the fiber bundle and either be drawn out by an external vacuum source, or simply vented. As the gas is flowing through the bundle of fibers, a flow of venous (unoxygenated) blood may be introduced into the bladder through the blood inlet nozzle. The venous blood may enter the bladder in a pulsatile flow manner (i.e., with each beat of the heart) and may flow through spaces between (and across the surfaces of) the various fibers comprising the bundle. Since the fibers comprising the bundle are made of a microporous or nonporous material, the partial pressures of oxygen and carbon dioxide in the blood (and in the oxygen-rich gas) enables oxygen to pass from the fibers into the blood, while simultaneously enabling carbon dioxide to pass form the blood into the fibers where it is then drawn out and exhausted. Accordingly, this diffusion process aids in transforming venous blood into arterial (oxygenated) blood. The newly oxygenated blood may then flow through a plurality of spaced openings in a central portion of the hollow core around which the fibers are wound. The natural elasticity of the bladder may cause the bladder to expand as blood is injected into the interior of the bladder. This same elasticity may then cause the bladder to contract, forcing the oxygenated blood to flow upwards through core and out of a blood outlet nozzle for return to the heart and/or lung for circulation. The elasticity of the bladder enables the compliant artificial lung to pump blood out in a continuous flow, thus simulating the function of an anatomical lung.

One advantage provided by the invention is the ability of the compliant artificial lung to be utilized in an extracorporeal circuit (i.e., outside of the body) to assist a failing lung that has a favorable prognosis for recovery. The blood inlet and outlet nozzles of the compliant artificial lung may be placed either in series or in parallel with the pulmonary artery, or any other suitable blood vessel. If the compliant artificial lung is placed in series with the pulmonary artery or other vessel, blood may flow through both the compliant artificial lung and a natural lung. By contrast, if the compliant artificial lung is placed in parallel with the pulmonary artery or other vessel, blood may bypass the natural lung and flow only through the compliant artificial lung.

Another advantage of the invention is the ability of the compliant artificial lung to be implanted within the body to assist or replace a failing lung that has a poor prognosis for recovery. Similar to the embodiment described above, the blood inlet and outlet nozzles of the compliant artificial lung may be placed either in series or in parallel with the pulmonary artery, or any other suitable blood vessel.

Yet another advantage of the invention is that, regardless of whether the compliant artificial lung is implanted or part of an extracorporeal circuit, a separate blood pump may not be required as the flexible bladder of the artificial lung is designed to be sufficiently compliant to withstand the volume, pressure, and flow-rate characteristics of blood pumped within a natural range of the stroke volume of the heart. In this regard, the compliant artificial lung simulates the function of an anatomical lung by accepting pulsatile blood flow from the heart while providing a continuous flow of oxygenated blood as output.

Still yet another advantage of the invention is the ability of the compliant artificial lung to be scalable in size for use with patients of varying size (e.g., children and adults).

Another advantage of the invention is that, according to a different embodiment, a compliant artificial lung of one size may be provided such that it may be small enough for implantation in a child. A plurality of these smaller, compliant artificial lungs may then be assembled for implantation within an adult.

Yet another advantage of the invention is the ability of the compliant artificial lung to be adapted for use with non-human mammals, in addition to humans.

Still yet another advantage of the invention is the ability to provide a compliant artificial lung without a flexible elastic bladder. A bladder may be unnecessary if the bundle of fibers used to transport oxygen-rich gas further comprises a plurality of elastic spacer threads (or any other volume-compensating mechanism) disposed between the fibers situated around the outer periphery of the bundle, such that the resulting arrangement of fibers and spacer threads defines a sealed chamber (having vacant spaces between interior fibers of the bundle for blood flow) that is flexible enough to withstand the volume, pressure, and flow-rate characteristics of blood pumped within a natural range of the stroke volume of the heart.

These and other objects, features, and advantages of the invention will be apparent through the detailed description of the preferred embodiments and the drawings attached hereto. It is also to be understood that both the foregoing general description and the following detailed description are exemplary and not restrictive of the scope of the invention.

### BRIEF DESCRIPTION OF THE DRAWINGS

FIG. 1 is an illustration of a compliant artificial lung, according to an embodiment of the invention.

FIG. 2 is an illustration of a membrane oxygenator and a flexible housing (or bladder) comprising a compliant artificial lung, according to an embodiment of the invention.

FIG. 3 illustrates a sectional view of a membrane oxygenator adapted for use with a compliant artificial lung, according to an embodiment of the invention.

FIG. 4 is an exploded, perspective view of a membrane oxygenator adapted for use with a compliant artificial lung, according to an embodiment of the invention.

FIG. 5 illustrates a sectional view of a compliant artificial lung, according to an embodiment of the invention.

FIG. 6 illustrates an enlarged, sectional view of the gas permeable, microporous fiber bundle illustrated in FIGS. 2 and 4, according to an embodiment of the invention.

FIG. 7 illustrates a compliant artificial lung, according to an embodiment of the invention.

### DETAILED DESCRIPTION OF PREFERRED EMBODIMENTS

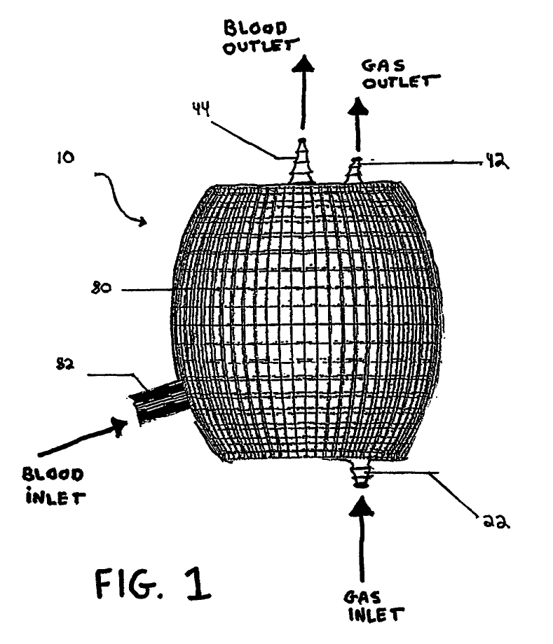

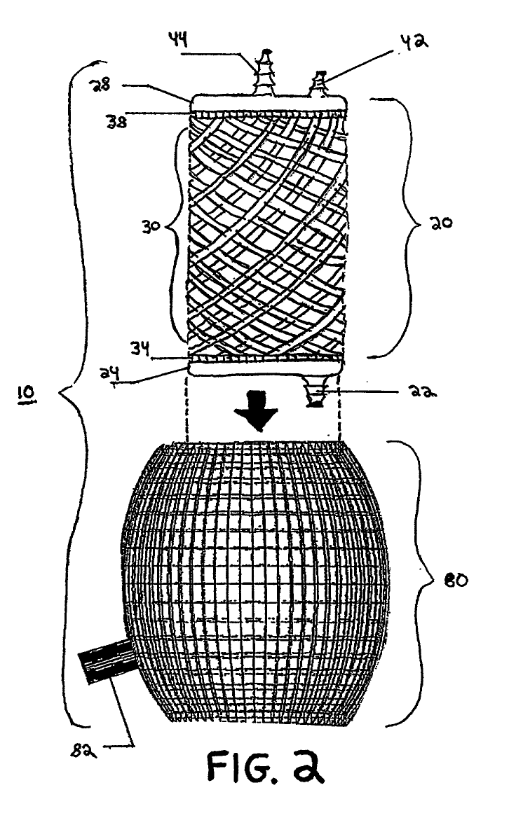

As illustrated in FIGS. 1 and 2, a compliant artificial lung 10 for extrapulmonary gas transfer is provided. According to an embodiment of the invention, compliant artificial lung 10 may comprise a membrane oxygenator 20 adapted to be disposed within a flexible housing or bladder 80. Bladder 80 may define a hollow chamber for receiving blood introduced via a blood inlet nozzle 82. Membrane oxygenator 20 may comprise a bundle 30 of microporous or nonporous, hollow fibers adapted to transport an oxygen-rich gas. When membrane oxygenator 20 is disposed within bladder 80, as described in greater detail below, venous blood introduced through blood inlet nozzle 82 may flow through spaces between (and across the surfaces of) the various fibers comprising bundle 30. The surface contact between the venous blood and the microporous or non-porous fibers of bundle 30 facilitates the gas-exchanging function of transporting oxygen to the blood while removing carbon dioxide. This aids in transforming venous blood into arterial blood.

According to an embodiment of the invention, bladder 80 may be fabricated from any suitable elastic material (e.g., silicone, polyurethane, medical grade rubber, latex, or others) having sufficient flexibility and elasticity to withstand the volume, pressure, and flow-rate characteristics of blood pumped within a natural range of the stroke volume of the heart. As a rough approximation, the range of cardiac output for a human heart (including both adults and children) may average between one-hundred milliliters of blood per minute and twelve liters of blood per minute. As such, bladder 80 may be designed to absorb or withstand, as an average, a flow of approximately one-hundred milliliters of blood to twelve liters of blood per minute without providing any appreciable resistance for the heart. In this regard, bladder 80 may act as a “natural” pump, enabling compliant artificial lung 10 to more closely approximate the function of an anatomical lung by accepting pulsatile blood flow from the heart, while providing a continuous flow of oxygenated blood as output.

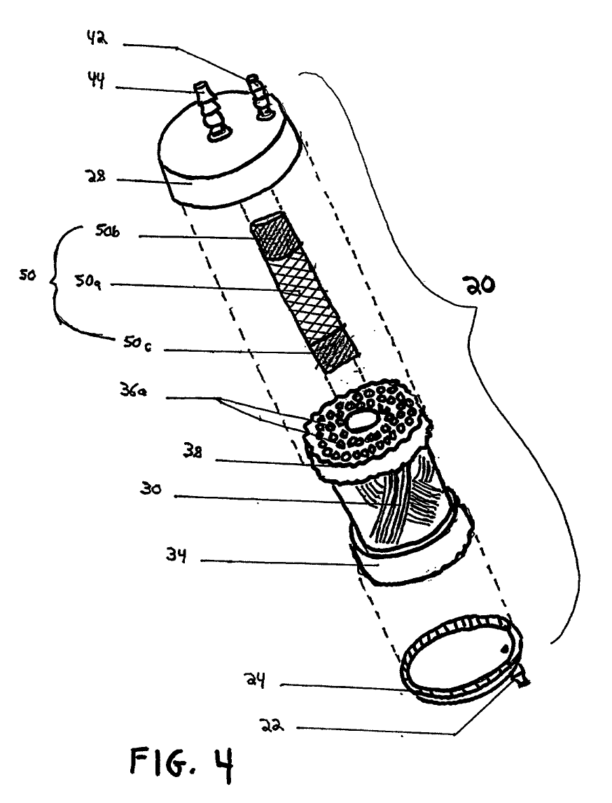

Referring now to FIGS. 2-4, a description of the various components comprising membrane oxygenator 20 is provided. According to an embodiment of the invention, the plurality of microporous, or nonporous hollow fibers comprising bundle 30 may be disposed about a hollow core 50. The fibers may preferably be comprised of any gas-permeable, biocompatible material including, but not limited to porous or non-porous nylon membrane, silicon membrane, polyolefin membrane, polyester membrane, or polypropylene membrane. The fibers of bundle 30 may be spirally wound around core 50 to form a plurality of layers. In particular, the direction of the spiral winding may change, layer by layer, to create a mesh of fibers arranged at oblique angles. This arrangement of bundle 30 is best illustrated in FIGS. 2 and 4. Winding the fibers in such a manner results in the creation of vacant spaces between the fibers that may permit the flow of blood therethrough. In addition, the mesh of fibers resulting from the spiral winding may also act to resist the inward pressure that a flow of blood may exert on the fibers.

According to various embodiments of the invention, additional arrangements, geometries, or configurations of hollow fibers may also be used. For example, rather than winding hollow fibers about a hollow core in a spiral fashion, square or triangular sheets of fibers, or other shaped sheets of fibers, may be used. Other configurations may exist and will be described below.

According to an embodiment of the invention, the respective upper ends 36a of the fibers comprising bundle 30 may be secured in place by dipping them in a potting material. The potting material may comprise a molten resin potting material, a polyurethane potting material, or any other suitable potting material. Preferably, the respective upper ends 36a of the fibers may be arranged such that their openings are spaced equidistantly. Once the resin potting material has dried, a layer of the resin potting material may be cut-off, resulting in a planar, upper potting layer 38. As best illustrated in FIG. 4, upper potting layer 38 comprises a planar surface, wherein the respective openings of the respective upper ends 36a of the fibers comprising bundle 30 are spaced equidistantly. To provide additional support for bundle 30, an upper core portion 50b of core 50 may extend through, and be anchored in, upper potting layer 38.

Similarly, the respective lower ends 36b of the fibers comprising bundle 30 may be spaced equidistantly and secured in place by dipping them in a potting material. Once the resin potting material has dried, a layer of the resin potting material may be cut-off, resulting in a planar, lower potting layer 34. Lower potting layer 34 comprises a planar surface, wherein the respective openings of the respective lower ends 36b of the fibers comprising bundle 30 are spaced equidistantly. A lower core portion 50c of core 50 may be securely embedded within lower potting layer 34 to provide additional structural support.

According to an embodiment of the invention, a lower annular chamber 24 may extend from lower potting layer 34. A gas inlet nozzle 22 may be integral with lower annular chamber 24, and may be used as a conduit for introducing gas from a gas blender or other source (not illustrated) into lower annular chamber 24, and into the respective lower ends 36b of the fibers of bundle 30. According to an embodiment of the invention, lower annular chamber 24 and gas inlet nozzle 22 may be fabricated from any suitable surgical grade, bio-compatible materials, such as, for example, stainless steel, ceramics, titanium, or plastics. Other materials may be used.

At the upper end of membrane oxygenator 20, an upper annular chamber 28 may extend from upper potting layer 38. Gas supplied to fiber bundle 30, via gas inlet nozzle 22, may emerge through the respective upper ends 36a of the fibers and into upper annular chamber 28. According to an embodiment of the invention, the gas may then be vented through a gas outlet nozzle 42 which may be integral with upper annular chamber 28. Alternatively, gas outlet nozzle 42 may be connected to a vacuum source (not illustrated) designed to assist in drawing the gas through fiber bundle 30. Upper annular chamber 28 and gas outlet nozzle 42 may also be fabricated from any suitable surgical grade, bio-compatible materials, such as stainless steel, ceramics, titanium, plastics, or other materials.

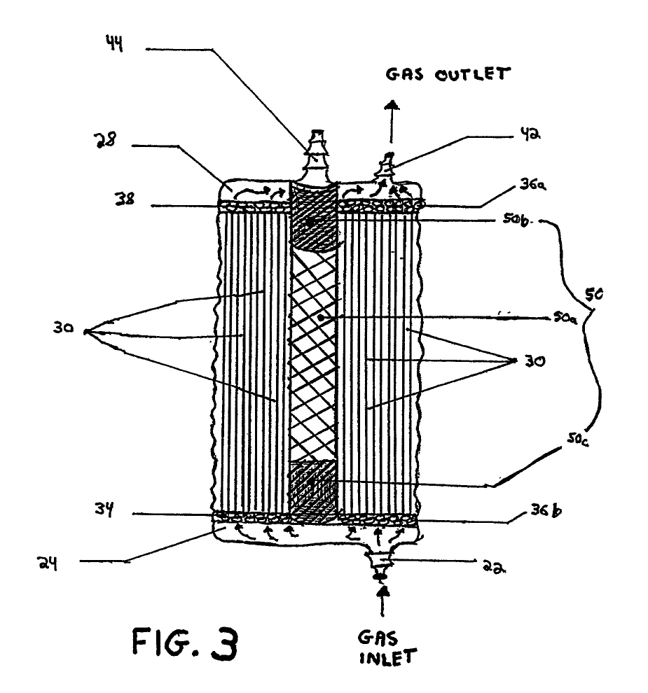

As illustrated in FIGS. 3-5, hollow core 50 may comprise an upper core portion 50b and lower core portion 50c as previously described, as well as a central core portion 50a. Core 50 may, according to an embodiment of the invention, be comprised of any suitable surgical grade, bio-compatible materials. Central core portion 50a may comprise a plurality of openings along its surface designed to enable blood (flowing through the vacant spaces between the fibers) to enter the interior of hollow core 50. Lower core portion 50c, although hollow, contains no openings along its outer surface and terminates within the middle of lower potting layer 34. This prevents blood from flowing into lower annular chamber 24 where gas is introduced.

According to an embodiment of the invention, upper core portion 50b extends through both the upper potting layer 38 and upper annular chamber 28 and terminates in a blood outlet nozzle 44. Similar to lower core portion 50c, upper core portion 50b is hollow but contains no openings along its outer surface. This enables upper core potion to transport blood entering core 50 (through central core portion 50a) upwards and out through blood outlet nozzle 44 while preventing blood from flowing into upper annular chamber 28.

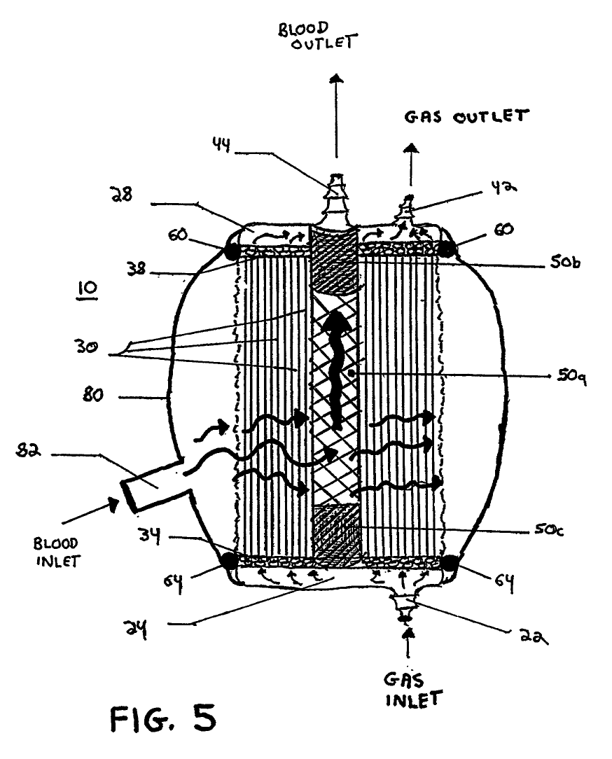

Referring now to FIG. 5, an illustration of compliant artificial lung 10 is provided wherein membrane oxygenator 20 is disposed within bladder 80. According to an embodiment of the invention, membrane oxygenator 20 may be secured within bladder 80 using an upper O-ring 60 and a lower O-ring 64. Upper O-ring 60 may, for example, be secured around upper potting layer 38 or upper annular chamber 28, or both. Lower O-ring 64 may, for example, be secured around lower potting layer 34 or lower annular chamber 24, or both.

In addition to functioning as a securing means, upper and lower O-rings (60, 64) may also serve as a sealant. When blood is introduced into bladder 80 through blood inlet nozzle 82, for example, a portion of it may flow through the vacant spaces between the layers of fiber bundle 30 and into the central core portion 50a of core 50. Some blood, however, may be dispersed across the fibers of bundle 30 without entering the central core portion 50a of core 50. O-rings (60, 64) may prevent this blood from seeping out the top and/or bottom openings of bladder 80. It should be recognized that O-rings (60,64) represent but one approach to securing and sealing membrane oxygenator 20 within bladder 80. Other suitable approaches may of course be utilized without deviating from the scope of the invention. It should also be understood that, in various embodiments, bladder 80 may completely enclose membrane oxygenator 20, while only providing openings that permit gas inlet nozzle 22, gas outlet nozzle 42, blood inlet nozzle 82, and blood outlet nozzle 44 to extend outward. In such an instance, O-rings or (any other suitable sealant) may be used to seal the protruding inlet and outlet nozzles.

Having provided a description of the various components comprising compliant artificial lung 10, a description of the various methods for using compliant artificial lung 10 is now provided. According to one embodiment of the invention, compliant artificial lung 10 may be utilized in an extracorporeal circuit (i.e., outside of the body) if a failing lung has a favorable prognosis for recovery. Blood inlet nozzle 82 and blood outlet nozzle 44 may be placed either in series or in parallel with the pulmonary artery or other suitable blood vessel. If compliant artificial lung 10 is placed in series with the pulmonary artery or other vessel, blood may flow through both compliant artificial lung 10 and a natural lung. By contrast, if compliant artificial lung 10 is placed in parallel with the pulmonary artery or other vessel, blood may bypass the natural lung and flow only through compliant artificial lung 10. Gas inlet nozzle 22 may be connected to a gas blender (not illustrated) or other source for providing an oxygen-rich gas mixture. Gas outlet nozzle 42 may either be connected to vacuum source (not illustrated) for drawing the oxygen-rich gas mixture through fiber bundle 30, or else left unconnected and permitted to vent. A blood-heating device (not illustrated) may also be connected to the extracorporeal circuit to heat the re-oxygenated blood (output from compliant artificial lung 10) prior to being re-introduced into the body. Unlike many conventional extracorporeal circuits, however, a blood pump may not be needed as compliant artificial lung 10 is designed to be sufficiently compliant to withstand the volume, pressure, and flow-rate characteristics of blood pumped within a natural range of the stroke volume of the heart.

According to another embodiment of the invention, compliant artificial lung 10 may be implanted within the body if a failing lung has a poor prognosis for recovery. In such an instance, any of the external surfaces of compliant artificial lung 10 may be coated with any suitable biocompatible materials. Similar to the embodiment described above, blood inlet nozzle 82 and blood outlet nozzle 44 may be placed either in series or in parallel with the pulmonary artery or other suitable blood vessel. Gas inlet nozzle 22 may be connected to a gas blender (not illustrated) or other source, external to the body, for providing an oxygen-rich gas mixture. Gas outlet nozzle 42 may be connected to a vacuum source (not illustrated) located external to the body. For instances in which compliant artificial lung 10 is implanted within the body, a patient may likely be required to remain connected to the external gas blender and vacuum source. This may require a patent to remain somewhat stationary unless the external devices are made to be portable.

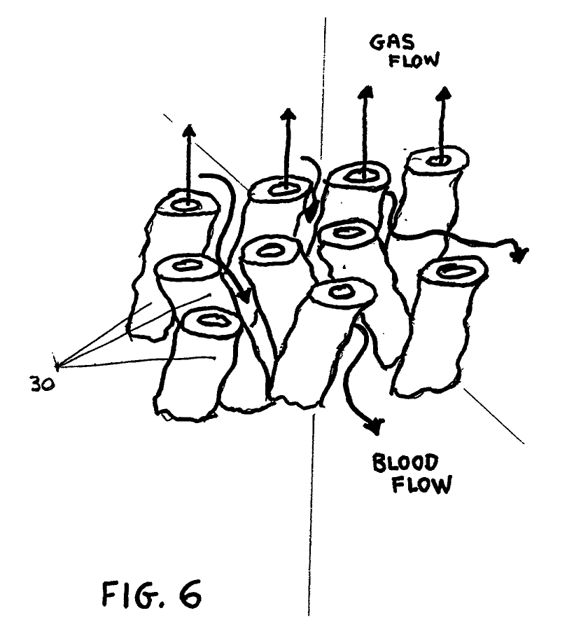

Regardless of whether compliant artificial lung 10 is implanted or part of an extracorporeal circuit, it may function in the same manner. According to an embodiment of the invention illustrated in FIG. 5, an oxygen-rich gas may be introduced into lower annular chamber 24 via gas inlet nozzle 22. The gas may enter the respective lower ends 36b of fiber bundle 30, travel through the fiber bundle 30, and be drawn out of upper annular chamber 28 and through gas outlet nozzle 42 by an external vacuum source. As the gas is flowing according to the process described above, a flow of venous (unoxygenated) blood may be introduced into bladder 80 through blood inlet nozzle 82. The venous blood may enter bladder 80 in a pulsatile flow manner (i.e., with each beat of the heart) and may flow through spaces between (and across the surfaces of) the various fibers comprising bundle 30. FIG. 6 depicts an enlarged, sectional view of the gas permeable, microporous fiber bundle 30 and illustrates how blood may through spaces between (and across the surfaces of) the various fibers as gas is being transported through them. Since the fibers comprising bundle 30 are made of a microporous or non-porous material, the partial pressures of oxygen and carbon dioxide in the blood (and in the oxygen-rich gas) enables oxygen to pass from the fibers into the blood, while simultaneously enabling carbon dioxide to pass form the blood into the fibers where it is then drawn out and exhausted. Accordingly, this diffusion process aids in transforming venous blood into arterial (oxygenated) blood. The newly oxygenated blood may then flow through the openings in the central portion 50a of hollow core 50. The natural elasticity of bladder 80 may cause bladder 80 to expand as blood is injected into the interior of the bladder. This same elasticity causes bladder 80 to then contract, forcing the oxygenated blood to flow upwards through core 50 and out of blood outlet nozzle 44 for return to the heart for circulation. The elasticity of bladder 80 enables compliant artificial lung 10 to pump blood out in a continuous flow, thus simulating the function of an anatomical lung.

The foregoing description of the invention along with the accompanying drawing figures set forth embodiments of one implementation of the invention. It should be recognized that the invention may implemented in any number of various other embodiments. For example, according to one embodiment, membrane oxygenator 20 and bladder 80 may be constructed as an integral unit.

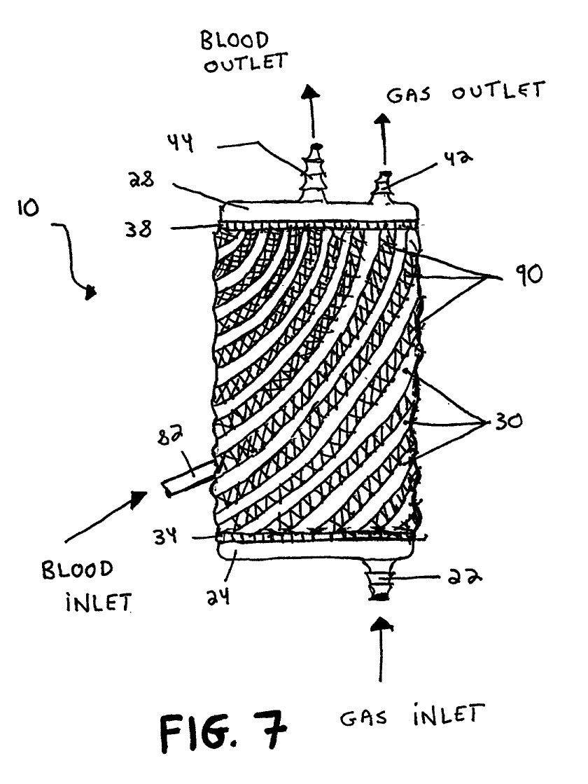

According to an embodiment of the invention illustrated in FIG. 7, a compliant artificial lung 10 may be provided without a flexible elastic bladder. A bladder may be unnecessary if fiber bundle 30 further comprises a plurality of elastic spacer threads 90 (or any other volume-compensating mechanism) disposed between the fibers situated around the outer periphery of the bundle, such that the resulting arrangement of fibers 30 and spacer threads 90 defines a sealed chamber (having vacant spaces between interior fibers of the bundle for blood flow) that is flexible enough to withstand the volume, pressure, and flow-rate characteristics of blood pumped within a natural range of the stroke volume of the heart.

Other embodiments, uses and advantages of the invention will be apparent to those skilled in the art from consideration of the specification and practice of the invention disclosed herein. The specification should be considered exemplary only, and the scope of the invention is accordingly intended to be limited only by the following claims.

What is claimed is:

1. A compliant artificial lung for extrapulmonary gas exchange, comprising:
a bladder defining a hollow chamber and having a blood inlet, the bladder being sufficiently compliant to expand when receiving a pulsatile flow of blood pumped within a natural range of the stroke volume of the heart;
a membrane oxygenator adapted to be centrally disposed within the bladder; the membrane oxygenator comprising a bundle of gas permeable hollow fibers for transporting a mixture of oxygen-rich gas, the fibers being disposed about a hollow core, and adapted to be in fluid contact with the blood received in the bladder to enable oxygen from the gas mixture to permeate through the hollow fibers and into the blood while simultaneously enabling carbon dioxide in the blood to permeate into the hollow fibers for removal from the fibers; and
wherein the compliance of the bladder enables it to naturally contract forcing oxygenated blood to flow through a plurality of openings in a surface of the hollow core and out of a blood outlet in a continuous flow.

2. The compliant artificial lung of claim 1, wherein the blood inlet and blood outlet are connected in series with a pulmonary artery.

3. The compliant artificial lung of claim 1, wherein the blood inlet and blood outlet are connected in parallel with a pulmonary artery.

4. The compliant artificial lung of claim 1, wherein the bundle of gas permeable hollow fibers are spirally wound around the hollow core to form a plurality of layers arranged at oblique angles with respect to one another.

5. The compliant artificial lung of claim 1, wherein each of the gas permeable hollow fibers comprising the bundle have upper ends and lower ends, each with an opening.

6. The compliant artificial lung of claim 5, wherein the respective upper ends of the fibers are secured in an upper layer of potting material, such that their respective upper openings are equidistantly spaced along a planar surface.

7. The compliant artificial lung of claim 6, wherein an upper annular chamber extends from the upper layer of potting material and further comprises a gas outlet.

8. The compliant artificial lung of claim 7, wherein the gas outlet is connected to a vacuum source for drawing exhaust gas out of the respective upper ends of the fibers, into the upper annular chamber, and out of the gas outlet.

9. The compliant artificial lung of claim 8, wherein the gas outlet comprises an exhaust valve.

10. The compliant artificial lung of claim 5, wherein the respective lower ends of the fibers are secured in a lower layer of potting material, such that their respective lower openings are equidistantly spaced along a planar surface.

11. The compliant artificial lung of claim 10, wherein a lower annular chamber extends from the lower layer of potting material and further comprises a gas inlet.

12. The compliant artificial lung of claim 11, wherein the gas inlet is connected to a gas blender used for introducing an oxygen-rich gas mixture into the lower annular chamber, and into the respective lower ends of the fibers.

13. The compliant artificial lung of claim 1, wherein the membrane oxygenator is secured within the bladder using an upper O-ring and a lower O-ring.

14. The compliant artificial lung of claim 13, wherein the upper O-ring and lower O-ring are further used to prevent blood from seeping out of the bladder.

15. A method for compensating for a failing lung, the method comprising the steps of:
(a) providing a compliant artificial lung, comprising:
a bladder defining a hollow chamber and having a blood inlet, the bladder being sufficiently compliant to expand when receiving a pulsatile flow of blood pumped within a natural range of the stroke volume of the heart;
a membrane oxygenator adapted to be centrally disposed within the bladder, the membrane oxygenator comprising a bundle of gas permeable hollow fibers for transporting a mixture of oxygen-rich gas, the fibers being disposed about a hollow core, and adapted to be in fluid contact with the blood received in the bladder to enable oxygen from the gas mixture to permeate through the hollow fibers and into the blood while simultaneously enabling carbon dioxide in the blood to permeate into the hollow fibers for removal from the fibers; and
wherein the compliance of the bladder enables it to naturally contract forcing oxygenated blood to flow through a plurality of openings in a surface of the hollow core and out of a blood outlet in a continuous flow; and
(b) connecting the compliant artificial lung to a pulmonary artery or to the heart.

16. The method of claim 15, further comprising the step of connecting the compliant artificial lung in series with the pulmonary artery.

17. The method of claim 15, further comprising the step of connecting the compliant artificial lung in parallel with the pulmonary artery.

18. The method of claim 15, further comprising the step of implanting the compliant artificial lung within the body.

19. The method of claim 15, further comprising the step of utilizing the compliant artificial lung outside of the body in an extracorporeal circuit.

20. A method for manufacturing a compliant artificial lung for extrapulmonary gas exchange, the method comprising the steps of:
(a) providing a bladder, the bladder defining a hollow chamber and having a blood inlet, and being sufficiently compliant to expand when receiving a pulsatile flow of blood pumped within a natural range of the stroke volume of the heart; and
(b) providing a membrane oxygenator adapted to be centrally disposed within the bladder, the membrane oxygenator comprising a bundle of gas permeable hollow fibers for transporting a mixture of oxygen-rich gas, the fibers being disposed about a hollow core, and adapted to be in fluid contact with the blood received in the bladder to enable oxygen from the gas mixture to permeate through the hollow fibers and into the blood while simultaneously enabling carbon dioxide in the blood to permeate into the hollow fibers for removal from the fibers; and
wherein the compliance of the bladder enables it to naturally contract forcing oxygenated blood to flow through a plurality of openings in a surface of the hollow core and out of a blood outlet in a continuous flow.

21. A compliant artificial lung for extrapulmonary gas exchange, comprising:
a bundle of compliant, gas permeable hollow fibers disposed about a hollow core for transporting a mixture of oxygen-rich gas, the bundle comprising an outer layer of fibers and at least one inner layer of fibers;
a volume-compensating mechanism disposed between individual fibers of the outer layer of fibers, such that the bundle comprises a sealed chamber, and wherein the sealed chamber is sufficiently compliant to expand when receiving, through a blood inlet, a pulsatile flow of blood pumped within a natural range of the stroke volume of the heart; and
wherein the at least one inner layer of fibers is adapted to be in fluid contact with the blood received via the blood inlet to enable oxygen from the gas mixture to permeate through the hollow fibers and into the blood while simultaneously enabling carbon dioxide in the blood to permeate into the hollow fibers for removal from the fibers; and
wherein the compliance of the sealed chamber enables it to naturally contract forcing oxygenated blood to flow through a plurality of openings in a surface of the hollow core and out of a blood outlet in a continuous flow.

22. The compliant artificial lung of claim 21, wherein the volume-compensating mechanism comprises elastic thread.
